(19) United States
(12) Patent Application Publication
(10) Pub. No.: US 2001/0006665 A1
(43) Pub. Date: Jul. 5, 2001

(54) Composition comprising at least one wax and at least one oil

(76) Inventor: Frederic Auguste, Chevilly-Larue (FR)

Correspondence Address: FINNEGAN, HENDERSON, FARABOW,, GARRETT & DUNNER, L.L.P., 1300 I Street, N.W., Washington, DC, 20005, US

(21) Appl. No.: 09/725,192

(22) Filed: Nov. 29, 2000

(30) Foreign Application Priority Data
Nov. 30, 1999 (FR) 99 15098

(51) Int. Cl. A61K 9/107

(52) U.S. Cl. 424/401

(57) ABSTRACT

A composition for topical application comprising, in a physiologically acceptable medium, at least one liquid fatty phase comprising at least one oil and one wax having a melting point of less than 60° C. and a hardness at 20° C. of greater than or equal to 9.5 MPa.

The present invention relates to a composition for topical application comprising at least one liquid fatty phase which comprises at least one wax. For example, the inventive composition can be used in the cosmetics field. In one embodiment, the present invention is directed to a composition for making up or caring for at least one keratinous substance, such as at least one human keratinous substance. Non-limiting examples of at least one human keratinous substance include skin (including the lips), keratinous fibres, such as eyelashes, eyebrows and hair, and nails. In another embodiment, the present invention is directed to a process for making up or caring for at least one keratinous substance and yet another embodiment is directed to the use of the inventive composition for curving keratinous fibres.

According to the present invention, the composition can be provided in the form of a product for coating keratinous fibres, an eyeliner product, a make-up product (such as, for example, a make-up product for the body), a concealer product, an eyeshadow product, a blusher product, a foundation product, a lip product (such as, for example, a lipstick), a nail varnish product, a sun protection product, a skin colouring product or a styling product.

For example, the composition may be in the form of a product for coating keratinous fibres that may be chosen from make-up compositions, make-up bases, compositions to be applied to a make-up (also known as topcoats) and compositions for cosmetic treatments of at least one keratinous fibre. In one embodiment, the composition is in the form of a mascara product.

Generally, compositions for making up keratinous fibres, such as mascaras, comprise waxes which form a deposit which is intended to sheath the keratinous fibres after the application of the composition. It is known to incorporate the waxes in a liquid fatty phase, such as isododecane, in order to obtain a water-resistant make-up (also known as waterproof make-up).

However, when hard waxes, such as carnauba wax or candelilla wax, are incorporated in isododecane at a content of at least 10% by weight with respect to the total weight of the mixture of wax and liquid fatty phase, a significant thickening of the mixture may be observed, whereupon the mixture may then exhibit the consistency of a solid. The thickening of the mixture may make it difficult to use a large amount of the wax(es) in the composition. Furthermore, the thick composition may be difficult to apply to keratinous substances, such as eyelashes, and may have a tendency to form lumps. Thus, such make-up composition may not be homogeneous, and this lack of homogeneity may be harmful to the desired effect and may leave the user with a feeling of discomfort and/or unpleasantness.

Therefore, there remains a need for compositions for topical application which can comprise a large amount of at least one wax in at least one liquid fatty phase and which may avoid an excessive thickening of the composition.

The inventors have discovered that such a composition may be obtained by mixing wax(es) having a low melting point and a high hardness with at least one liquid fatty phase. According to the present invention, the composition can comprise up to 30% by weight of at least one wax without exhibiting a solid consistency. Further, the inventive composition may be easily applied to keratinous substances, and may result in a homogeneous make-up. Furthermore, when the inventive composition is applied to keratinous fibres, such as eyelashes, it may be possible to obtain significant curving of the keratinous fibres.

Specifically, one embodiment of the present invention is directed to a composition for topical application comprising, in a physiologically acceptable medium, at least one liquid fatty phase comprising at least one oil and at least one wax, wherein the at least one wax has a melting point of less than 60° C. and a hardness at 20° C. of greater than or equal to 9.5 MPa.

Another embodiment of the present invention is directed to a process for making up or non-therapeutically treating at least one keratinous substance comprising applying to the at least one keratinous substance a sufficient amount of the inventive composition as described above.

Yet another embodiment of the present invention is directed to a method for curving keratinous fibres comprising applying a sufficient amount of the inventive composition as defined above. In one embodiment, the keratinous fibres are eyelashes.

As used herein, the term “physiologically acceptable” refers to a medium compatible with the skin and/or keratinous fibres, such as a cosmetic medium.

According to the present invention, the at least one wax has a melting point generally ranging from 30° C. to 59° C., such as from 35° C. to 59° C. and further such as from 40° C. to 50° C.

Further, the at least one wax may have a hardness generally ranging from 9.5 MPa to 20 MPa, such as from 9.5 MPa to 15 MPa. For example, the at least one wax may have a hardness of greater than 10 MPa, such as ranging from 10to 20 MPa and further such as ranging from 10 to 12 MPa.

According to the present invention, the hardness of the wax is determined by the measurement of the compressive strength, measured at 20° C. using the texturometer sold under the name TA-XT2i by Rheo equipped with a stainless steel cylinder with a diameter of 2 mm which moves at the measurement rate of 0.1 mm/s and which penetrates into the wax to a penetration depth of 0.3 mm. To carry out the hardness measurement, the wax is melted at a temperature equal to the melting point of the wax +20° C. The molten wax is poured into a receptacle with a diameter of 30 mm and a depth of 20 mm. The wax is recrystallized at room temperature (25° C.) for 24 hours and then the wax is stored for at least one hour at 20° C. before carrying out the hardness measurement. The value of the hardness is the compressive strength measured divided by the surface area of the cylinder of the texturometer in contact with the wax.

The at least one wax according to the present invention may be chosen from compounds of formula (I):

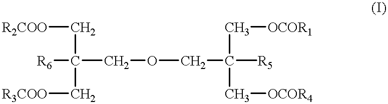

wherein:

R₁, R₂, R₃ and R₄, which may be identical or different, are each chosen from linear alkyl groups comprising from 13 to 19 carbon atoms;

R₅ and R₆, which may be identical or different, are each chosen from methyl groups, ethyl groups and propyl groups;

In one embodiment, R₁, R₂, R₃ and R₄, which may be identical or different, are each chosen from alkyl groups comprising from 15 to 17 carbon atoms. In another embodiment, R₁, R₂, R₃ and R₄ are each chosen from alkyl groups comprising 17 carbon atoms. In yet another embodiment, R₅ and R₆ are each a methyl group.

The compounds of formula (I) can be obtained from reaction of a polyol of formula (II)

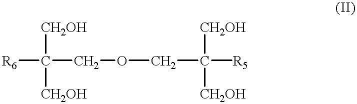

with a carboxylic acid R—COOH,

wherein R₅ and R₆, which may be identical or different, are each chosen from methyl groups, ethyl groups and propyl groups, and R is chosen from linear alkyl groups comprising from 13 to 19 carbon atoms.

A non-limiting example of compounds of formula (I) is the tetrastearate of di(1,1,1-trimethylol-propane), corresponding to formula (I) wherein R₁, R₂, R₃ and R₄ are simultaneously C₁₇ alkyl groups and R₅ and R₆ are simultaneously ethyl groups. This compound is sold under the name Hest 2T-4S by Heterene.

The at least one wax may also be chosen from waxes derived from hydrogenation of olive oil esterified with stearyl alcohol, such as, for example, the wax sold under the name Phytowax Olive 18 L 57 by Sophim.

The at least one wax according to the present invention is present in an amount generally ranging from 0.5% to 60% by weight with respect to the total weight of the composition, such as from 1 % to 40%, further such as from 2% to 30% and even further such as from 2% to 20%.

The at least one oil according to the present invention may be chosen from volatile oils and non-volatile oils. In one embodiment, the at least one oil is chosen from volatile oils.

As used herein, the term “volatile oil” refers to an oil capable of evaporating at room temperature from a support on which it has been applied (i.e. an oil having a measurable vapour pressure at room temperature).

The at least one oil suitable for use in the present invention may be chosen from oils which are volatile at room temperature and atmospheric pressure, such as, for example, oils having a vapour pressure at ambient pressure and room temperature >0 mm of Hg (>0 Pa), such as ranging from 10⁻³ mm of Hg to 300 mm of Hg (0.13 Pa to 40 000 Pa), provided that the boiling point is greater than 30° C. These volatile oils may form a film which demonstrates total “transfer-free” properties and good hold. These volatile oils may facilitate application of the composition to the skin, mucous membranes or superficial body growths. Accordingly to the present invention, these volatile oils can be chosen from volatile hydrocarbonaceous oils, volatile silicone oils and volatile fluorinated oils.

As used herein, the term “hydrocarbonaceous oil” refers to an oil comprising hydrogen atoms and carbon atoms, optionally further comprising at least one atom chosen from oxygen atoms, nitrogen atoms, sulphur atoms and phosphorus atoms. In one embodiment, the at least one oil is chosen from volatile hydrocarbonaceous oils comprising from 8 to 16 carbon atoms. Non-limiting examples of the aforementioned hydrocarbonaceous oils include C₈-C₁₆ isoalkanes (or isoparaffins) and branched C₈-C₁₆ esters, such as isododecane (also known as 2,2,4,4,6-pentamethyl-heptane), isodecane, isohexadecane, isohexyl neopentanoate and mixtures of any of the foregoing. Other volatile hydrocarbonaceous oils, such as oil distillates (for example those sold under the name Shell Solt by the company Shell) can also be used.

The volatile oils may also be chosen from volatile silicones, such as, for example, volatile cyclic silicone oils and volatile linear silicone oils. For example, the volatile cyclic silicone oils can be chosen from those having a viscosity of ≦8 centistokes (8×10⁻⁶ m²/s), such as octamethylcyclotetrasiloxane, decamethylcyclopentasiloxane and dodecamethylcyclohexasiloxane. For example, the volatile linear silicone oils can be chosen from octamethyltrisiloxane, heptamethylhexyltrisiloxane, heptamethyl-octyltrisiloxane and decamethyltetrasiloxane. Further, the volatile oils can be chosen from volatile fluorinated oils, such as nonafluoromethoxybutane and perfluoromethylcyclopentane.

The at least one volatile oil can be present in the composition according to the invention in an amount generally ranging from 1% to 99.5% by weight relative to the total weight of the composition, such as from 5% to 98% by weight, further such as from 30% to 70% by weight and even further such as from 40% to 60% by weight.

The composition according to the invention may comprise at least one non-volatile oil chosen from, for example, non-volatile hydrocarbonaceous, non-volatile silicone and non-volatile fluorinated oils.

For example, the non-volatile hydrocarbonaceous oils can be chosen from:

hydrocarbonaceous oils of animal origin, such as perhydrosqualene;

hydrocarbonaceous oils of vegetable origin, such as liquid triglycerides of fatty acids comprising from 4 to 10 carbon atoms, such as triglycerides of heptanoic acid, triglycerides of octanoic acid, sunflower, grape seed, sesame, maize, apricot, castor, avocado, olive, cereal germ, soybean, sweet almond, palm, rapeseed, cottonseed, hazelnut, macadamia and jojoba oils, triglycerides of caprylic/capric acids, such as those sold by the company Stéarineries Dubois and those sold under the names Miglyol 810, 812 and 818 by the company Dynamit Nobel and karite butter oil;

linear and branched hydrocarbons of mineral and synthetic origin, such as liquid petrolatum, polydecenes and hydrogenated polyisobutene, such as Parleam;

synthetic esters and ethers, such as oils of formula R₁₀COOR₁₁ wherein R₁₀ is chosen from residues of higher fatty acids comprising from 6 to 29 carbon atoms and R₁₁ is chosen from hydrocarbonaceous chains comprising from 3 to 30 carbon atoms, such as Purcellin oil, isopropyl myristate, isopropyl palmitate, butyl stearate, hexyl laurate, diisopropyl adipate, isononyl isononate, 2-ethylhexyl palmitate, 2-hexyldecyl laurate, 2-octyldecyl palmitate, 2-octyldodecyl myristate, 2-octyldodecyl lactate and polyol esters, such as propylene glycol dioctanoate, neopentyl glycol diheptanoate, diethylene glycol diisononanoate and pentaerythritol esters;

short-chain esters comprising from 3 to 8 carbon atoms, such as ethyl acetate, methyl acetate, propyl acetate, n-butyl acetate and isopentyl acetate;

fatty alcohols which are liquid at room temperature comprising at least one carbon chain which comprises from 12 to 26 carbon atoms wherein the at least one carbon chain is chosen from branched chains and unsaturated chains, such as octyldodecanol, isostearyl alcohol, oleyl alcohol, 2-hexyldecanol, 2-butyloctanol and 2-undecylpentadecanol;

higher fatty acids, such as myristic acid, palmitic acid, stearic acid, behenic acid, oleic acid, linoleic acid, linolenic acid and isostearic acid;

The non-volatile silicone oils which can be used in the composition according to the present invention can be oils of low viscosity, such as linear polysiloxanes with a degree of polymerization ranging from approximately 6 to approximately 2000. For example, the non-volatile silicone oils can be chosen from polydimethylsiloxanes (PDMSs) with a viscosity of greater than 10 mPa·s, phenyl dimethicones, phenyl trimethicones and polyphenylmethylsiloxanes.

If present, the non-volatile oils can be present in the composition according to the invention in an amount generally ranging up to 20% by weight relative to the total weight of the composition, such as from 0.1 to 20%, such as ranging up to 2% by weight and further such as from 0.1% to 2% by weight.

The composition according to the present invention can further comprise at least one additional wax different from the at least one wax described above. The at least one additional wax may be chosen from (i) waxes having a melting point of greater than or equal to 60° C. and (ii) waxes having a melting point of less than 60° C. and a hardness at 20° C. of less than 9.5 MPa. The at least one additional wax may have a melting point of greater than or equal to 40° C., such as from 60° C. to 110C. In one embodiment, the at least one liquid fatty phase comprises the at least one additional wax.

The at least one additional wax may be chosen from waxes of animal origin, waxes of vegetable origin, waxes of mineral origin and waxes of synthetic origin.

Examples of waxes of animal origin include beeswaxes, lanolin waxes and Chinese insect waxes.

Examples of waxes of vegetable origin include rice waxes, carnauba waxes, candelilla waxes, ouricury waxes, cork fibre waxes, sugar cane waxes, Japan waxes, sumach waxes and cottonseed waxes.

Examples of waxes of mineral origin include paraffin waxes, microcrystalline waxes, montan waxes and ozokerites.

Examples of waxes of synthetic origin include polyolefin waxes such as polyethylene waxes, waxes obtained by the Fischer-Tropsch synthesis, waxy copolymers and their esters and silicone waxes.

It is also possible to use hydrogenated oils of animal and vegetable origin which still correspond to the two physical characteristics of temperature and hardness mentioned above.

Examples of hydrogenated oils include hydrogenated oils which are derived from catalytic hydrogenation of fatty substances composed of at least one C₈-C₃₂ fatty chain chosen from linear and nonlinear C₈-C₃₂ fatty chains, hydrogenated sunfloweroil, hydrogenated castor oil, hydrogenated coconut oil, hydrogenated jojoba oil, hydrogenated lanolin oil and hydrogenated palm oil.

The at least one additional wax which can be used in the composition according to the present invention may be, for example, solid and stiff at a temperature of less than 50° C.

If present, the composition according to the present invention can comprise the at least one additional wax in an amount generally ranging up to 30% by weight relative to the total weight of the composition, such as from 0.1% to 30% and further such as from 0.5% to 25% by weight.

The composition according to the present invention can further comprise, in the at least one liquid fatty phase, at least one film-forming polymer. This at least one film-forming polymer may be soluble in the at least one liquid fatty phase or fat-soluble or may be dispersed in the form of particles in the at least one liquid fatty phase. This at least one film-forming polymer may confer good hold on the composition after application to keratinous substances.

As used herein, the term “film-forming polymer” refers to a polymer capable, alone, of forming an isolable film.

Mention may be made, by way of examples of fat-soluble film-forming polymers, of the polymers comprising at least one unit of formula (III):

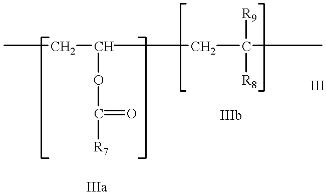

wherein:

R₇, which may be identical or different, are each chosen from linear and branched saturated hydrocarbonaceous chains comprising from 1 to 19 carbon atoms;

R₈, which may be identical or different, are each chosen from:

a) —O—CO—R₁₀, wherein R₁₀, which may be identical or different, are each chosen from linear and branched saturated hydrocarbonaceous chains comprising from 1 to 19 carbon atoms, with the proviso that said R₁₀ is different from said R₇;

b) —CH₂—R₁₁, wherein R₁₁, which may be identical or different, are each chosen from linear and branched saturated hydrocarbonaceous chains comprising from 5 to 25 carbon atoms;

c) —O—R₁₂, wherein R₁₂, which may be identical or different, are each chosen from saturated hydrocarbonaceous chains comprising from 2 to 18 carbon atoms; and

d) —CH₂—O—CO—R₁₃, wherein R₁₃, which may be identical or different, are each chosen from linear and branched saturated hydrocarbonaceous chains comprising from 1 to 19 carbon atoms,

R₉, which may be identical or different, are each chosen from (i) a hydrogen atom when said R₈ is chosen from —O—CO—R₁₀, —CH₂—R₁₁ and —CH₂—O—CO—R₁₃, and (ii) a methyl group when said R₈ is —CH₂—O—CO—R₁₃,

with the proviso that said copolymers of formula (III) comprise at least 15% by weight of at least one unit chosen from units (IIIa) and units (IIIb) derived from at least one monomer comprising at least one hydrocarbonaceous chain which comprises at least 7 carbon atoms, wherein said at least one hydrocarbonaceous chain is chosen from linear and branched saturated hydrocarbonaceous chains.

The copolymers of formula (III) may be derived from copolymerization of at least one vinyl ester (corresponding to the (IIIa) unit) and of at least one other monomer (corresponding to the (IIIb) unit) which can be chosen from, for example, α-olefins, alkyl vinyl ethers, allyl esters and methallyl esters.

When, in the (IIIb) unit, R₈ is chosen from the —CH₂—R₁₁, —O—R₁₂ and —CH₂—O—CO—R₁₃ groups as defined above, the copolymers of formula (III) can comprise from 50 mol % to 95 mol % of at least one (IIIa) unit and from 5 mol % to 50 mol % of at least one (IIIb) unit.

The copolymers of formula (III) may also be derived from copolymerization of at least one vinyl ester and of at least one other vinyl ester different from the first. In this case, these copolymers may comprise from 10 mol % to 90 mol % of at least one (IIIa) unit and from 10 mol % to 90 mol % of at least one (IIIb) unit in which R₈ represents a —O—CO—R₁₀ group.

Examples of vinyl esters resulting in the unit of formula (IIIa) or in the unit of formula (IIIb) in which R₈=—O—CO—R₁₀ include vinyl acetate, vinyl propionate, vinyl butanoate, vinyl octanoate, vinyl decanoate, vinyl laurate, vinyl stearate, vinyl isostearate, vinyl 2,2-dimethyl-octanoate and vinyl dimethylpropionate.

Examples of α-olefins resulting in the unit of formula (IIIb) in which R₈=—CH₂—R₁₁ include 1-octene, 1-dodecene, 1-octadecene, 1-eicosene and mixtures of α-olefins comprising from 22 to 28 carbon atoms.

Examples of alkyl vinyl ethers resulting in the unit of formula (IIIb) in which R₈= —O—R₁₂ include ethyl vinyl ether, n-butyl vinyl ether, isobutyl vinyl ether, decyl vinyl ether, dodecyl vinyl ether, cetyl vinyl ether and octadecyl vinyl ether.

Examples of allyl or methallyl esters resulting in the unit of formula (IIIb) in which R₈=—CH₂—O—CO—R₁₃ include allyl and methallyl acetates, propionates, dimethylpropionates, butyrates, hexanoates, octanoates, decanoates, laurates, 2,2-dimethylpentanoates, stearates and eicosanoates.

The copolymers of formula (III) can also be crosslinked using certain types of crosslinking agents, the aim of which may be to substantially increase their molecular weight.

This crosslinking may be carried out during the copolymerization and the crosslinking agents can be chosen from crosslinking agents of the vinyl type, crosslinking agents of the allyl type and crosslinking agents of the methallyl type. Examples of suitable crosslinking agents include tetraallyloxyethane, divinylbenzene, divinyl octanedioate, divinyl dodecanedioate and divinyl octadecanedioate.

Examples of copolymers of formula (III) which can be used in the composition according to the invention, include the copolymers: vinyl acetate/allyl stearate, vinyl acetate/vinyl laurate, vinyl acetate/vinyl stearate, vinyl acetate/octadecene, vinyl acetate/octadecyl vinyl ether, vinyl propionate/allyl laurate, vinyl propionate/vinyl laurate, vinyl stearate/1-octadecene, vinyl acetate/1-dodecene, vinyl stearate/ethyl vinyl ether, vinyl propionate/cetyl vinyl ether, vinyl stearate/allyl acetate, vinyl 2,2-dimethyl-octanoate/vinyl laurate, allyl 2,2-dimethyl-pentanoate/vinyl laurate, vinyl dimethyl-propionate/vinyl stearate, allyl dimethyl-propionate/vinyl stearate, vinyl propionate/vinyl stearate, crosslinked with 0.2% of divinylbenzene, vinyl dimethylpropionate/vinyl laurate, crosslinked with 0.2% of divinylbenzene, vinyl acetate/octadecyl vinyl ether, crosslinked with 0.2% of tetraallyloxyethane, vinyl acetate/allyl stearate, crosslinked with 0.2% of divinylbenzene, vinyl acetate/1-octa-decene, crosslinked with 0.2% of divinylbenzene, and allyl propionate/allyl stearate, crosslinked with 0.2% of divinylbenzene.

Examples of fat-soluble film-forming polymers include fat-soluble homopolymers such as those derived from homo-polymerization of a vinyl ester comprising from 9 to 22 carbon atoms, homopolymers derived from homo-polymerization of an alkyl acrylate wherein the alkyl group comprises from 10 to 20 carbon atoms and homopolymers derived from homopolymerization of an alkyl methacrylate wherein the alkyl group comprises from 10 to 20 carbon atoms.

For example, fat-soluble homopolymers can be chosen from poly(vinyl stearate), poly(vinyl stearate) crosslinked using at least one of divinylbenzene, diallyl ether and diallyl phthalate, poly(stearyl (meth)acrylate), poly(vinyl laurate) and poly(lauryl (meth)acrylate), it being possible for these poly(meth)acrylates to be crosslinked using at least one of ethylene glycol and tetraethylene glycol dimethacrylate.

The fat-soluble copolymers and homopolymers defined above are known and are disclosed in Application FR-A-2,232,303 and U.S. Pat. No. 3,937,811, the disclosures of which are incorporated herein by reference. Further, the fat-soluble copolymers and homopolymers may have a weight-average molecular weight ranging from 2,000 to 500,000, such as from 4,000 to 200,000.

Examples of fat-soluble film-forming polymers which can be used in the present invention include polyalkylenes other than the polyolefin wax defined in a) (such as copolymers derived from monomers chosen from C₂-C₂₀ alkenes, such as polybutene), alkylcelluloses wherein the alkyl group, which may be identical or different, are each chosen from saturated and unsaturated, linear and branched C₁ to C₈ alkyl groups (such as ethylcellulose and propylcellulose) and vinylpyrrolidone (VP) copolymers (such as copolymers derived from (i) at least one monomer of vinylpyrrolidone and (ii) at least one monomer chosen from C₂ to C₄₀ alkenes, such as a C₃ to C₂₀ alkene). Mention may be made, by way of examples of VP copolymers which can be used in the present invention, of VP/vinyl acetate copolymers, VP/ethyl methacrylate copolymers, butylated polyvinylpyrrolidone (PVP)copolymers, VP/ethyl methacrylate/methacrylic acid copolymers, VP/eicosene copolymers, VP/hexadecene copolymers, VP/triacontene copolymers, VP/styrene copolymers and VP/acrylic acid/lauryl methacrylate copolymers.

If present, the at least one film-forming polymer can be present in the composition in an amount generally ranging from 0.1% to 20% by weight relative to the total weight of the composition, such as from 0.5% to 15% by weight.

The composition according to the present invention can further comprise at least one stable dispersion of particles of at least one polymeric system, wherein said at least one polymeric system is chosen from polymeric systems which are able to form a film and polymeric systems which are unable to form a film, and wherein the particles are stabilized at the surface in the at least one liquid fatty phase. Obviously, the inventive composition can comprise a mixture of a stable dispersion of (i) a first polymeric system which is able to form a film and (ii) a second polymeric system which is unable to form a film.

The particles of the polymeric system may have a size generally ranging from 5 nm to 600 nm, such as from 50 nm to 250 nm. Further, these particles may be substantially spherical in shape.

As used herein, the term “polymeric system which is able to form a film” refers to a polymeric system capable of forming an isolable film at room temperature (25° C). The first film-forming polymeric system may have a low glass transition temperature (Tg 1) of less than or equal to the temperature of the skin, such as a low glass transition temperature of less than or equal to approximately 40° C. For example, the Tg 1 may generally range from approximately −100° C. to approximately 40° C., such as from −10° C. to 30° C.

As used herein, the term “polymeric system which is unable to form a film” refers to a polymeric system which does not make it possible to form an isolable film at room temperature (25° C.). The second polymeric system which is unable to form a film may make it possible, however, in combination with the liquid fatty phase, to form a continuous and homogeneous coating on keratinous substances. The second polymeric system which is unable to form a film may have a glass transition temperature (Tg 2) of greater than approximately 40° C., such as less than or equal to approximately 300° C. For example, the Tg 2 may generally range from approximately 45° C. to approximately 150° C.

The Tg (glass transition temperature) may be measured by DSC (Differential Scanning Calorimetry) according to ASTM Standard D3418-97.

According to one embodiment, the at least one polymeric system can comprise at least one polymer wherein the at least one polymeric system has the characteristics of the polymeric system which are described above.

When the at least one polymer alone does not make it possible to obtain the characteristics of the at least one polymeric system which are mentioned above, it is possible to add at least one compound to modify at least one of the properties of the at least one polymer, in order to obtain a desired polymeric system. Consequently, according to another embodiment, it is possible to add, to the at least one polymer, at least one plasticizing agent which may make it possible to obtain at least one polymeric system having the characteristics as described above. In this case, the at least one polymeric system comprises a mixture of the at least one polymer and the at least one plasticizing agent.

The at least one plasticizer may be chosen from plasticizers conventionally used in the field of application, such as plasticizers capable of being solvents of the polymer.

The at least one polymer in dispersion in the at least one liquid fatty phase may have any nature. Examples of the at least one polymer which can be used include radical polymers, polycondensates and polymers of natural origin. The at least one polymer may be chosen by a person skilled in the art according to their properties.

The at least one polymer in dispersion which may be used in the composition of the present invention may have a weight-average molecular weight generally ranging from 2,000 to 10,000,000.

As used herein, the term “radical polymer” refers to a polymer derived from polymerization of at least one monomer with unsaturation, such ethylenic unsaturation, wherein the at least one monomer is capable of homo-polymerizing (unlike polycondensates). The radical polymers may be chosen from vinyl polymers and vinyl copolymers, such as acrylic polymers.

The vinyl polymers may be derived from polymerization of at least one monomer with ethylenic unsaturation comprising at least one group chosen from acid groups, ester groups and amide groups.

Examples of monomers comprising at least one acid group include α,β-ethylenic unsaturated carboxylic acids, such as acrylic acid, methacrylic acid, crotonic acid, maleic acid and itaconic acid. In one embodiment, the monomers may be chosen from (meth)acrylic acid and crotonic acid and in another embodiment the monomers are (meth)acrylic acid.

The monomers comprising at least one ester group may be chosen from esters of (meth)acrylic acid (also known as (meth)acrylates) such as alkyl (meth)acrylates, (such as C₁-C₂₀ alkyl (meth)acrylates, and further such as C₁-C₈ (meth)acrylates), aryl (meth)acrylates (such as C₆-C₁₀ aryl (meth)acrylates) and hydroxyalkyl (meth)acrylates (such as C₂-C₆ hydroxyalkyl (meth)acrylates).

Examples of alkyl (meth)acrylates include methyl (meth)acrylate, ethyl (meth)acrylate, butyl (meth)acrylate, isobutyl (meth)acrylate, 2-ethylhexyl (meth)acrylate and lauryl (meth)acrylate.

Examples of hydroxyalkyl (meth)acrylates include hydroxyethyl (meth)acrylate and 2-hydroxypropyl (meth)acrylate.

Examples of aryl (meth)acrylates include benzyl acrylate and phenyl acrylate.

Further examples of radical polymers include copolymers of (meth)acrylic acid and of alkyl (meth)acrylates, such as C₁-C₄ alkyl (meth)acrylate. Still further examples include methyl acrylates optionally copolymerized with acrylic acid.

Examples of monomers comprising at least one amide group include (meth)acrylamides, such as the N-alkyl(meth)acrylamides (such as N-(C₂-C₁₂ alkyl)(meth)acrylamides which may, for example, be chosen from N-ethylacrylamide, N-(t-butyl)acrylamide and N-octylacrylamide) and further such as N,N-di(C₁-C₄ alkyl)(meth)acrylamides.

The vinyl polymers may also be derived from polymerization of at least one monomer with ethylenic unsaturation comprising at least one amine group, wherein the at least one amine group may be chosen from amine groups in the free form, partially neutralized amine groups, completely neutralized amine groups, partially quaternized amine groups and completely quaternized amine groups. For example, the at least one monomer with ethylenic unsaturation comprising at least one amine group may be chosen from dimethylaminoethyl (meth)acrylate, (dimethylaminoethyl)methacrylamide, vinylamine, vinylpyridine and diallyidimethylammonium chloride.

The vinyl polymers may also be derived from homo- or co-polymerization of at least one monomer chosen from vinyl esters and styrene monomers. In this case, the at least one monomer can be polymerized with at least one monomer chosen from monomers comprising at least one acid group, monomers comprising at least one ester group and monomers comprising at least one amide group, such as those mentioned above. Examples of vinyl esters include vinyl acetate, vinyl propionate, vinyl neodecanoate, vinyl pivalate, vinyl benzoate and vinyl t-butylbenzoate.

Examples of styrene monomers include styrene and alpha-methylstyrene.

The list of monomers given is not limiting and it is possible to use any monomer known to a person skilled in the art coming within the categories of acrylic and vinyl monomers (including monomers modified with a silicone chain).

Other examples of vinyl monomers include:

N-vinylpyrrolidone, vinylcaprolactam, N-(C₁-C₆ alkyl)vinylpyrroles, vinyloxazoles, vinylthiazoles, vinylpyrimidines and vinylimidazoles, and

olefins, such as ethylene, propylene, butylene, isoprene and butadiene.

The vinyl polymer can be crosslinked using at least one difunctional monomer which may comprise at least two ethylenic unsaturations, such as, for example, ethylene glycol dimethacrylate and diallyl phthalate.

With such dispersions of polymer particles, it may be possible to grade, as desired, the size of the polymer particles and to adjust their “polydispersity” in size during the synthesis. It may thus be possible to obtain particles of very small size, which are invisible to the naked eye when they are in the composition and when they are applied to the skin or the lips. This might not be possible with pigments in particulate form, their constitution not allowing the mean size of the particles to be varied.

Non-limiting examples of the at least one polymer in dispersion include: polyurethanes, polyurethane-acrylics, polyureas, polyurea-polyurethanes, polyester-polyurethanes, polyether-polyurethanes, polyesters, polyesteramides, polyesters comprising at least one fatty chain, alkyds, silicone polymers, and fluoropolymers.

The at least one polymer can be used in a sufficient amount to produce a continuous and homogeneous film, deposited on at least one keratinous substance such as keratinous fibres, which is at least one of glossy, non-sticky and having at least one transfer-free property.

The at least one polymer dispersed in the at least one liquid fatty phase can be present in a total amount generally ranging from 2% to 60% by weight relative to the total weight of the composition, such as from 4% to 40% by weight, and further such as from 5% to 30% by weight.

The polymer particles may be stabilized at the surface, during the polymerization, by virtue of at least one stabilizing agent which may be, for example, chosen from block polymers, graft polymers and random polymers. The stabilization can be carried out by any known means, such as, for example, by direct addition of the at least one stabilizing agent during the polymerization.

The at least one stabilizing agent may be present in the mixture before polymerization. However, it is also possible to add it continuously, in particular when the monomers are also added continuously.

The composition can comprise the at least one stabilizing agent in an amount generally ranging from 2% to 30% by weight relative to the starting mixture of at least one monomer, such as 5% to 20% by weight.

When the at least one stabilizing agent is chosen from graft and block polymers, the synthesis solvent may be chosen such that at least a portion of the grafts (or blocks) of the at least one stabilizing polymer is soluble in said solvent, the other portion of the grafts (or blocks) not being soluble therein. The at least one stabilizing polymer used during the polymerization may be soluble, or dispersible, in the synthesis solvent. Furthermore, the at least one stabilizing agent may be chosen for which the insoluble grafts (or blocks) exhibit a degree of affinity for the polymer formed during the polymerization.

Examples of graft polymers include silicone polymers grafted with at least one hydrocarbonaceous chain and hydrocarbonaceous polymers grafted with at least one silicone chain.

Graft copolymers having, for example, an insoluble backbone of polyacrylic type with soluble grafts of poly(12-hydroxystearic acid) type are also suitable.

Further examples of suitable graft and block copolymers include graft and block copolymers comprising at least one block of polyorganosiloxane type and at least one block of radical polymer type, such as graft copolymers of acrylic/silicone type, which can be employed, for example, when the non-aqueous medium is a silicone medium.

The at least one stabilizing agent may also be chosen from graft and block copolymers comprising at least one block of polyorganosiloxane type and at least one block of polyether type. The polyorganopolysiloxane block may be, for example, chosen from polydimethylsiloxanes and poly(C₂-C₁₈)alkylmethylsiloxanes; the polyether block may, for example, be chosen from poly(C₂-C₁₈ alkylenes), such as polyoxyethylene and polyoxypropylene. Examples of the at least one stabilizing agent include dimethicone copolyols and (C₂-C₁₈)alkyl methicone copolyols, such as, for example, dimethicone copolyol sold under the name “Dow Corning 3225C” by Dow Corning and the lauryl methicone copolyol sold under the name “Dow Corning Q2-5200” by Dow Corning.

Further examples of graft and block copolymers include copolymers comprising at least one block derived from polymerization of at least one ethylenic monomer with at least one optionally conjugated ethylenic bond, such as ethylene, butadiene and isoprene, and at least one block of styrene polymer type. When the at least one ethylenic monomer comprises several optionally conjugated ethylenic bonds, the residual ethylenic unsaturations after the polymerization may be hydrogenated. Thus, in a known way, the polymerization of isoprene may result, after hydrogenation, in the formation of an ethylene-propylene block and the polymerization of butadiene may result, after hydrogenation, in the formation of an ethylene-butylene block. Examples of these block copolymers include copolymers of “diblock” and “triblock” type of the polystyrene/polyisoprene or polystyrene/polybutadiene type (such as those sold under the name of “Luvitol HSB” by BASF), copolymers of “diblock” and “triblock” type of the polystyrene/copoly(ethylene-propylene) type (such as those sold under the name of “Kraton” by Shell Chemical Co.) and copolymers of “diblock” and “triblock” type of the polystyrene/copoly(ethylene-butylene) type.

Further examples of graft and block copolymers comprising at least one block resulting from the polymerization of at least one ethylenic monomer (such as ethylene and isobutylene) and at least one block of acrylic polymer type (such as methyl methacrylate) include poly(methyl methacrylate)/polyisobutylene bi- and triblock copolymers and graft copolymers with a poly(methyl methacrylate) backbone and polyisobutylene grafts.

Examples of graft and block copolymers comprising at least one block derived from polymerization of at least one ethylenic monomer and at least one block of polyether type (such as a polyoxy(C₂-C₁₈ alkylene), such as polyoxyethylene and polyoxypropylene) include polyoxyethylene/ polybutadiene bi- and triblock copolymers and polyoxyethylene/polyisobutylene bi- and triblock copolymers.

It is also possible to employ copolymers of C₁-C₄ alkyl (meth)acrylates and of C₈-C₃₀ alkyl (meth)acrylates, such as stearyl methacrylate/methyl methacrylate copolymers.

When the at least one stabilizing agent is chosen from random polymers, it may be chosen so that it comprises a sufficient amount of groups to render it soluble in the envisaged synthesis solvent.

When the synthesis solvent is non-polar, the at least one stabilizing agent may be chosen from polymers which allow the most complete possible covering of the particles, in which case several chains of stabilizing polymers may then be adsorbed on one particle of polymer obtained by polymerization.

Examples of such an at least one stabilizing agent may include graft polymers and block polymers, so as to have a better interfacial activity. This is because the blocks or grafts which are insoluble in the synthesis solvent may contribute a bulkier covering to the surface of the particles.

When the liquid synthesis solvent comprises at least one silicone oil, the at least one stabilizing agent may be chosen from graft and block copolymers comprising at least one block of polyorganosiloxane type and at least one block of a polymer chosen from radical polymers, polyethers and polyesters, such as polyoxy(C₂-C₁₈)alkylene blocks (such as polyoxypropylenated blocks and polyoxyethylenated blocks).

When the at least one liquid fatty phase does not comprise a silicone oil, the stabilizing agent may be chosen from:

(a) graft and block copolymers comprising at least one block of polyorganosiloxane type and at least one block chosen from radical polymers, polyethers and polyesters;

(b) copolymers derived from (i) at least one monomer chosen from C₁-C₄ alkyl acrylates and C₁-C₄ alkyl methacrylates and (ii) at least one monomer chosen from C₈-C₃₀ alkyl acrylates and C₈-C₃₀ alkyl methacrylates; and

(c) graft and block copolymers comprising at least one block derived from the polymerization of at least one ethylenic monomer with at least one conjugated ethylenic bond and at least one block chosen from blocks of vinyl polymers, acrylic polymers, polyethers and polyesters.

It is, of course, possible to use combinations of the at least one stabilizing agent described above. Further, diblock polymers may be used as the at least one stabilizing agent.

Depending on the application, it is possible to choose to use dispersions of polymers, which are able or unable to form films, in volatile and/or non-volatile oils.

Polymer dispersions in a liquid fatty phase are disclosed in the documents EP-A-749 746, EP-A-749 747, EP-A-923 928 and EP-A-930 060, the disclosures of which are incorporated herein by reference.

The composition according to the invention may further comprise at least one silicone gum. The at least one silicone gum may be chosen from polysiloxanes with a high molecular mass, ranging from 200,000 to 1,000,000, and having a viscosity of greater than 500,000 mPa·s. The at least one silicone gum can be used alone or as a mixture with a solvent, such as a polydimethylsiloxane oil and polyphenylsiloxane oil.

If present, the at least one silicone gum can be present in the composition in an amount generally ranging up to 2% by weight relative to the total weight of the composition, such as from 0.1% to 1% by weight.

The composition according to the invention may further comprise at least one thickening agent for thickening the liquid fatty phase. The at least one thickening agent may be chosen from organomodified clays which are clays treated with compounds chosen, for example, from quaternary amines and tertiary amines. Mention may be made, as organomodified clays, of organomodified bentonites, such as those sold under the name “Bentone 34” by the company Rheox, and organomodified hectorites, such as those sold under the names “Bentone 27” and “Bentone 38” by the company Rheox.

If present, the at least one thickening agent can be present in an amount ranging from 0.5% to 10% by weight relative to the total weight of the composition, such as from 1% to 6% by weight.

The composition may further comprise at least one coloring material. The at least one coloring material can be chosen, for example, from pulverulent compounds and fat-soluble dyes. If present, the at least one coloring material can be present in an amount generally ranging from 0.01% to 30% by weight relative to the total weight of the composition. The pulverulent compounds may be chosen from pigments, pearlescent agents and fillers commonly used in cosmetic or dermatological compositions. If present, the pulverulent compounds may be present in an amount generally ranging from 0.1% to 25% by weight relative to the total weight of the composition, such as from 1 % to 20%.

The pigments may be white or coloured, inorganic and/or organic. Examples of inorganic pigments include titanium dioxide, which is optionally surface treated, zirconium oxides, cerium oxides, as well as iron and chromium oxides, manganese violet, ultramarine blue, chromium hydrate and ferric blue. Examples of organic pigments include carbon black, pigments of D & C type, and lakes based on at least one of cochineal carmine, barium, strontium, calcium and aluminium.

The pearlescent pigments may be chosen from white pearlescent pigments, such as mica covered with at least one of titanium oxide and bismuth oxychloride, coloured pearlescent pigments, such as titanium dioxide-coated mica covered with iron oxides, titanium dioxide-coated mica covered with at least one of ferric blue and chromium oxide and titanium dioxide-coated mica covered with an organic pigment of the above mentioned type and pearlescent pigments based on bismuth oxychloride.

The fillers can be chosen from those well known to a person skilled in the art which are commonly used in cosmetic compositions.

The composition can further comprise at least one suitable additive generally used in such compositions, such as preservatives, fragrances, sunscreen agents, agents for combating free radicals, moisturizing agents, vitamins, proteins, ceramides and plasticizers.

Of course, a person skilled in the art will take care to choose this or these optional additional compounds and/or their amount so that the advantageous properties of the composition according to the invention are not, or not substantially, detrimentally affected by the envisaged addition.

The composition according to the invention may be anhydrous. The composition may comprise up to 10% by weight of water, such as from 0.1% to 10% by weight, with respect to the total weight of the composition, wherein the water forms an aqueous phase. The optional aqueous phase can comprise at least one cosmetically or dermatologically acceptable additive and may comprise at least one additional film-forming polymer in the form of particles dispersed in the aqueous phase.

The composition according to the invention may be provided in the form of a fluid, a gel, a semi-solid, a soft paste or a solid, such as a stick. The form of the cosmetic composition may be prepared according to the methods usual in the field under consideration.

The composition according to the invention may be used, for example, for making up and/or cosmetically treating keratinous substances. The make-up composition may be, for example, any of the following: an eyeliner, a mascara, a foundation, an eyeshadow, a blusher, a lipstick, a concealer or a product for making up the body of the temporary or semi-permanent tattooing type. The cosmetic treatment composition may be, for example, a composition for caring for the face, neck, hands and/or body; it may also constitute an anti-sun or self-tanning composition.

The invention also pertains to processes for making and using the disclosed compositions. Such processes may include, for example, the following.

A process for caring for, making up or treating at least one keratinous substance comprising applying to said at least one keratinous substance a composition comprising at least one liquid fatty phase comprising at least one oil and at least one wax having a melting point of less than 60° C. and a hardness at 20° C. of greater than or equal to 9.5 MPa.

A process for non-therapeutic treatment of at least one keratinous substance comprising applying to said at least one keratinous substance a composition comprising at least one liquid fatty phase comprising at least one oil and at least one wax having a melting point of less than 60° C. and a hardness at 20° C. of greater than or equal to 9.5 MPa.

A process for curving keratinous fibres, such as eyelashes, comprising applying to said keratinous fibres a composition comprising at least one liquid fatty phase comprising at least one oil and at least one wax having a melting point of less than 60° C. and a hardness at 20° C. of greater than or equal to 9.5 MPa.

Other than in the operating example, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective testing measurements. The following examples are intended to illustrate the invention without limiting the scope as a result. The percentages are given on a weight basis.

The invention is illustrated in more detail in the following examples.

### EXAMPLE 1

### Comparative Study

The consistency of 4 waxes (including 2 according to the invention) mixed at various contents in isododecane was studied.

Wax 1: tetrastearate of di(1,1,1-trimethylol-propane), sold under the name Hest 2T-4S by Heterene

Wax 2: wax sold under the name Phytowax Olive 18 L 57 by Sophim

Wax 3: carnauba wax

Wax 4: candelilla wax

These tested waxes had a melting point and a hardness as follows:

| Wax | Wax 1 (I) | Wax 2 (I) | Wax 3 (OI) | Wax 4 (OI) |
|---|---|---|---|---|
| Melting point (° C.) | 46 | 58 | 82 | 64 |
| Hardness (MPa) | 10.3 | 10 | 7 | 9.6 |

I: invention;
OI: outside the invention

The hardness of each wax was measured using the TA-XT2i texturometer from Rheo.

The following consistencies were observed (the contents were expressed as percentage by weight with respect to the total weight of the wax/isododecane mixture):

| Wax | Wax 1 (I) | Wax 2 (I) | Wax 3 (OI) | Wax 4 (OI) |
|---|---|---|---|---|
| 10% | liquid | liquid | solid | solid |
| 20% | thick gel | liquid | solid | solid |
| 30% | pasty | pasty | solid | solid |
| 50% | solid | solid | solid | solid |

It was found that the waxes 1 and 2 according to the invention could be mixed with isododecane up to a content of at least 30% by weight without the mixture being solid, whereas the waxes 3 and 4 of the prior art formed a solid mixture from the 10% content by weight.

### EXAMPLE 2

A mascara was prepared with the following composition:

| | | |
|---|---|---|
| Tetrastearate of di(1,1,1-trimethylolpropane) | 20 | g |
| (Hest 2T-4S from Heterene) | | |
| Vinyl acetate/allyl stearate (65/35) copolymer | 7 | g |
| (Mexomère PQ from Chimex) | | |
| Quaternium-18 bentonite | 5 | g |
| Propylene carbonate | 1.6 | g |
| Black iron oxide | 5 | g |
| Isododecane q.s. for | 100 | g |

The composition was easily applied to the eyelashes and conferred very good curving on the latter.

### EXAMPLE 3

A mascara was prepared with the following composition:

| | | |
|---|---|---|
| Wax (Phytowax Olive 18 L 57 from Sophim) | 17 | g |
| Vinyl acetate/allyl stearate (65/35) copolymer (Mexomère PQ | 7 | g |
| from Chimex) | | |
| Quaternium-18 bentonite | 5 | g |
| Propylene carbonate | 1.6 | g |
| Black iron oxide | 5 | g |
| Isododecane q.s. for | 100 | g |

After application of the mascara to the eyelashes, very good curving of the eyelashes was obtained.

### EXAMPLE 4

A mascara was prepared with the following composition:

| | | |
|---|---|---|
| Polymer dispersion of Example 5 | 10 | g |
| Wax (Phytowax Olive 18 L 57 from Sophim) | 15 | g |
| Vinyl acetate/allyl stearate (65/35) copolymer (Mexomère PQ | 2.2 | g |
| from Chimex) | | |
| Poly(vinyl laurate) (Mexomère PP from Chimex) | 0.75 | g |
| Rice starch | 0.84 | g |
| Bentonite | 5.32 | g |
| Propylene carbonate | 1.74 | g |
| Pigments | 4.6 | g |
| Preservatives q.s | | |
| Isododecane q.s. for | 100 | g |

### EXAMPLE 5 of Polymer Dispersion

A dispersion of non-crosslinked copolymer of methyl acrylate and of acrylic acid, 25 in a 95/5 ratio, in isododecane was prepared according to the method of Example 7 of the document EP-A-749 747. A dispersion of poly(methyl acrylate/acrylic acid) particles stabilized at the surface in isododecane by a polystyrene/copoly(ethylene-propylene) diblock copolymer sold under the name of Kraton G1701 (Shell) was thus obtained with a dry matter level of 24.2% by weight and a mean size of the particles of 180 nm and a Tg of 20° C. This copolymer was able to form a film at room temperature (25° C.).

What is claimed is:

1. A composition comprising at least one liquid fatty phase comprising:
(a) at least one oil; and
(b) at least one wax having a melting point of less than 60° C. and a hardness at 20° C. of greater than or equal to 9.5 MPa,
in a physiologically acceptable medium.

2. A composition according to claim 1, wherein said at least one wax has a melting point ranging from 30° C. to 59° C.

3. A composition according to claim 2, wherein said at least one wax has a melting point ranging from 35° C. to 59° C.

4. A composition according to claim 3, wherein said at least one wax has a melting point from 40° C. to 50° C.

5. A composition according to claim 1, wherein said at least one wax has a hardness at 20° C. ranging from 9.5 MPa to 20 MPa.

6. A composition according to claim 5, wherein said at least one wax has a hardness at 20° C. ranging from 9.5 MPa to 15 MPa.

7. A composition according to claim 6, wherein said at least one wax has a hardness at 20° C. ranging from 10 MPa to 12 MPa.

8. A composition according to claim 1, wherein said at least one wax is chosen from compounds of formula (I): 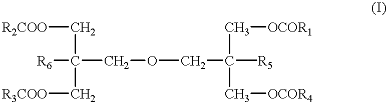
wherein:
R₁, R₂, R₃ and R₄, which may be identical or different, are each chosen from linear alkyl groups comprising from 13 to 19 carbon atoms, and
R₅ and R₆, which may be identical or different, are each chosen from methyl groups, ethyl groups and propyl groups.

9. A composition according to claim 8, wherein said R₁, R₂, R₃ and R₄, which may be identical or different, are each chosen from linear alkyl groups comprising from 15 to 17 carbon atoms.

10. A composition according to claim 9, wherein said R₁, R₂, R₃ and R₄ are each chosen from linear alkyl groups comprising 17 carbon atoms.

11. A composition according to claim 8, wherein said R₅ and R₆ are each a methyl group.

12. A composition according to claim 1, wherein said at least one wax is a tetrastearate of di(1,1,1-trimethylolpropane).

13. A composition according to claim 1, wherein said at least one wax is chosen from waxes derived from hydrogenation of olive oil esterified with stearyl alcohol.

14. A composition according to claim 1, wherein said at least one wax is present in an amount ranging from 0.5% to 60% by weight relative to the total weight of said composition.

15. A composition according to claim 14, wherein said at least one wax is present in an amount ranging from 1% to 40% by weight relative to the total weight of said composition.

16. A composition according to claim 15, wherein said at least one wax is present in an amount ranging from 2% to 30% by weight relative to the total weight of said composition.

17. A composition according to claim 16, wherein said at least one wax is present in an amount ranging from 2% to 20% by weight relative to the total weight of said composition.

18. A composition according to claim 1, wherein said at least one oil is chosen from volatile oils and non-volatile oils.

19. A composition according to claim 18, wherein said at least one oil is chosen from volatile oils.

20. A composition according to claim 19, wherein said volatile oils are chosen from volatile hydrocarbonaceous oils, volatile silicone oils and volatile fluorinated oils.

21. A composition according to claim 20, wherein said volatile oils are chosen from isoparaffins comprising from 8 to 16 carbon atoms.

22. A composition according to claim 20, wherein said volatile oils are chosen from octamethylcyclotetrasiloxane, decamethylcyclopentasiloxane, dodecamethylcyclohexasiloxane, octamethyltrisiloxane, heptamethylhexyltrisiloxane, heptamethyloctyltrisiloxane and decamethyltetrasiloxane.

23. A composition according to claim 20, wherein said volatile oils are chosen from nonafluoromethoxybutane and perfluoromethylcyclopentane.

24. A composition according to claim 19, wherein said volatile oils are present in an amount ranging from 1% to 99.5% by weight relative to the total weight of said composition.

25. A composition according to claim 24, wherein said volatile oils are present in an amount ranging from 5% to 98% by weight relative to the total weight of said composition.

26. A composition according to claim 25, wherein said volatile oils are present in an amount ranging from 30% to 70% by weight relative to the total weight of said composition.

27. A composition according to claim 26, wherein said volatile oils are present in an amount ranging from 40% to 60% by weight relative to the total weight of said composition.

28. A composition according to claim 18, wherein said non-volatile oils are chosen from non-volatile hydrocarbonaceous oils, non-volatile silicone oils and non-volatile fluorinated oils.

29. A composition according to claim 28, wherein said non-volatile hydrocarbonaceous oils are chosen from:
hydrocarbonaceous oils of animal origin;
hydrocarbonaceous oils of vegetable origin;
linear and branched hydrocarbons of mineral origin and linear and branched hydrocarbons of synthetic origin;
synthetic esters and synthetic ethers;
short-chain esters comprising from 3 to 8 carbon atoms;
fatty alcohols which are liquid at room temperature comprising at least one carbon chain which comprises from 12 to 26 carbon atoms, wherein said at least one carbon chain is chosen from branched chains and unsaturated chains; and
higher fatty acids.

30. A composition according to claim 28, wherein said non-volatile oils are present in an amount ranging up to 20% by weight relative to the total weight of said composition.

31. A composition according to claim 28, wherein said non-volatile oils are present in an amount ranging from 0.1% to 20% by weight relative to the total weight of said composition.

32. A composition according to claim 30, wherein said non-volatile oils are present in an amount ranging up to 2% by weight relative to the total weight of said composition.

33. A composition according to claim 31, wherein said non-volatile oils are present in an amount ranging from 0.1% to 2% by weight relative to the total weight of said composition.

34. A composition according to claim 1, further comprising at least one additional wax chosen from (i) waxes having a melting point of greater than or equal to 60° C. and (ii) waxes having a melting point of less than 60° C. and a hardness at 20° C. of less than 9.5 MPa.

35. A composition according to claim 34, wherein said at least one additional wax has a melting point ranging from 60° C. to 110° C.

36. A composition according to claim 34, wherein said at least one additional wax is chosen from waxes of animal origin, waxes of vegetable origin and waxes of synthetic origin.

37. A composition according to claim 34, wherein said at least one additional wax is present in an amount ranging up to 30% by weight relative to the total weight of said composition.

38. A composition according to claim 34, wherein said at least one additional wax is present in an amount ranging from 0.5% to 25% by weight relative to the total weight of said composition.

39. A composition according to claim 34, wherein said at least one liquid fatty phase comprises said at least one additional wax.

40. A composition according to claim 1, wherein said at least one liquid fatty phase further comprises at least one film-forming polymer.

41. A composition according to claim 40, wherein said at least one film-forming polymer is chosen from copolymers comprising at least one unit of formula (III): 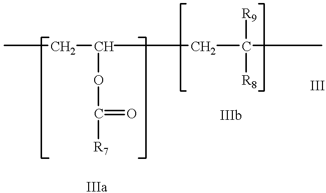
wherein:
R₇, which may be identical or different, are each chosen from linear and branched saturated hydrocarbonaceous chains comprising from 1 to 19 carbon atoms;
R₈, which may be identical or different, are each chosen from:
a) —O—CO—R₁₀, wherein R₁₀, which may be identical or different, are each chosen from linear and branched saturated hydrocarbonaceous chains comprising from 1 to 19 carbon atoms, with the proviso that said R₁₀ is different from said R₇;
b) —CH₂—R₁₁, wherein R₁₁, which may be identical or different, are each chosen from linear and branched saturated hydrocarbonaceous chains comprising from 5 to 25 carbon atoms;
c) —O—R₁₂, wherein R₁₂, which may be identical or different, are each chosen from saturated hydrocarbonaceous chains comprising from 2 to 18 carbon atoms; and
d) —CH₂—O—CO—R₁₃, wherein R₁₃, which may be identical or different, are each chosen from linear and branched saturated hydrocarbonaceous chains comprising from 1 to 19 carbon atoms,
R₉, which may be identical or different, are each chosen from (i) a hydrogen atom when said R₈ is chosen from —O—CO—R₁₀, —CH₂—R₁₁ and —CH₂—O—CO—R₁₃, and (ii) a methyl group when said R₈ is —CH₂—O—CO—R₁₃,
with the proviso that said copolymers of formula (III) comprise at least 15% by weight of at least one unit chosen from units (IIIa) and units (IIIb) derived from at least one monomer comprising at least one hydrocarbonaceous chain which comprise at least 7 carbon atoms, wherein said at least one hydrocarbonaceous chain is chosen from linear and branched saturated hydrocarbonaceous chains.

42. A composition according to claim 40, wherein said at least one film-forming polymer is a homopolymer chosen from homopolymers derived from a vinyl ester comprising from 9 to 22 carbon atoms and homopolymers derived from an alkyl (meth)acrylate wherein the alkyl group comprises from 10 to 20 carbon atoms.

43. A composition according to claim 40, wherein said at least one film-forming polymer is chosen from copolymers derived from monomers chosen from C₂-C₂₀ alkenes, alkylcelluloses wherein the alkyl group, which may be identical or different, are each chosen from saturated and unsaturated, linear and branched C₁ to C₈ alkyl groups and vinylpyrrolidone copolymers.

44. A composition according to claim 43, wherein said vinylpyrrolidone copolymers are chosen from copolymers derived from (i) at least one monomer chosen from vinylpyrrolidones and (ii) at least one monomer chosen from C₂ to C₄₀ alkenes.

45. A composition according to claim 40, wherein said at least one film-forming polymer is fat-soluble and is present in an amount ranging from 0.1% to 20% by weight relative to the total weight of the composition.

46. A composition according to claim 45, wherein said at least one film-forming polymer is fat-soluble and is present in an amount ranging from 0.5% to 15% by weight relative to the total weight of the composition.

47. A composition according to claim 1, further comprising at least one dispersion of particles of at least one polymeric system, wherein said at least one polymeric system is chosen from polymeric systems which can form a film and polymeric systems which cannot form a film and wherein said particles are stabilized at the surface in said at least one liquid fatty phase.

48. A composition according to claim 47, wherein said polymeric systems which can form a film have a glass transition temperature of less than or equal to about 40° C.

49. A composition according to claim 48, wherein said polymeric systems which can form a film have a glass transition temperature ranging from about −100° C. to about 40° C.

50. A composition according to claim 49, wherein said polymeric systems which can form a film have a glass transition temperature ranging from about −10° C. to about 30° C.

51. A composition according to claim 47, wherein said polymeric systems which cannot form a film have a glass transition temperature of greater than about 40° C. and less than or equal to 300° C.

52. A composition according to claim 51, wherein said polymeric systems which cannot form a film have a glass transition temperature ranging from about 45° C. to about 150° C.

53. A composition according to claim 47, wherein said at least one polymeric system further comprises at least one plasticizing agent.

54. A composition according to claim 47, wherein said at least one polymeric system comprises at least one polymer chosen from radical polymers, polycondensates and polymers of natural origin.

55. A composition according to claim 54, wherein said radical polymers are chosen from vinyl polymers and vinyl copolymers.

56. A composition according to claim 54, wherein said at least one polymer has a weight-average molecular weight ranging from 2 000 to 10 000 000.

57. A composition according to claim 47, wherein said at least one polymeric system comprises at least one polymer chosen from polyurethanes, polyurethane-acrylics, polyureas, polyurea-polyurethanes, polyester-polyurethanes, polyether-polyurethanes, polyesters, polyesteramides, polyesters comprising at least one fatty chain, alkyds, acrylic polymers, acrylic copolymers, vinyl polymers, vinyl copolymers, silicone polymers and fluoropolymers.

58. A composition according to claim 54, wherein said at least one polymer is present in an amount ranging from 2% to 60% by weight relative to the total weight of said composition.

59. A composition according to claim 58, wherein said at least one polymer is present in an amount ranging from 4% to 40% by weight relative to the total weight of said composition.

60. A composition according to claim 59, wherein said at least one polymer is present in an amount ranging from 5% to 30% by weight relative to the total weight of said composition.

61. A composition according to claim 47, wherein said particles are stabilized with at least one stabilizing agent chosen from block polymers, graft polymers and random polymers.

62. A composition according to claim 61, wherein said at least one stabilizing agent is chosen from silicone polymers grafted with at least one hydrocarbonaceous chain, hydrocarbonaceous polymers grafted with at least one silicone chain, graft and block copolymers comprising at least one block of polyorganosiloxane type and at least one block of radical polymer type, graft and block copolymers comprising at least one block of polyorganosiloxane type and at least one block of polyether type, copolymers derived from at least one monomer chosen from C₁-C₄ alkyl (meth)acrylates, copolymers derived from at least one monomer chosen from C₈-C₃₀ alkyl (meth)acrylates, graft and block copolymers comprising at least one block derived from at least one ethylenic monomer comprising at least one optionally conjugated ethylenic bond and at least one block of styrene polymer type, graft and block copolymers comprising at least one block derived from at least one ethylenic monomer and at least one block of acrylic polymer type and graft and block copolymers comprising at least one block derived from at least one ethylenic monomer and at least one block of polyether type.

63. A composition according to claim 62, wherein said at least one stabilizing agent is chosen from graft and block copolymers comprising at least one block derived from at least one ethylenic monomer comprising at least one optionally conjugated ethylenic bond and at least one block of styrene polymer type.

64. A composition according to claim 1, further comprising at least one silicone gum.

65. A composition according to claim 64, wherein said at least one silicone gum is present in an amount ranging up to 2% by weight relative to the total weight of said composition.

66. A composition according to claim 65, wherein said at least one silicone gum is present in an amount ranging from 0.1% to 1% by weight relative to the total weight of said composition.

67. A composition according to claim 1, further comprising at least one thickening agent for thickening said at least one liquid fatty phase.

68. A composition according to claim 67, wherein said at least one thickening agent is present in an amount ranging from 0.5% to 10% by weight relative to the total weight of said composition.

69. A composition according to claim 68, wherein said at least one thickening agent is present in an amount ranging from 1% to 6% by weight relative to the total weight of said composition.

70. A composition according to claim 1, further comprising at least one coloring material.

71. A composition according to claim 70, wherein said at least one coloring material is chosen from pulverulent compounds and fat-soluble dyes.

72. A composition according to claim 71, wherein said pulverulent compounds are chosen from pigments, pearlescent agents and fillers.

73. A composition according to claim 70, wherein said at least one coloring material is present in an amount ranging from 0.01% to 30% by weight relative to the total weight of said composition.

74. A composition according to claim 1, further comprising at least one suitable additive chosen from preservatives, fragrances, sunscreen agents, agents for combating free radicals, moisturizing agents, vitamins, proteins, ceramides and plasticizers.

75. A composition according to claim 1, wherein said composition is anhydrous.

76. A composition according to claim 1, wherein said composition further comprises water in an amount less than 10% by weight relative to the total weight of said composition, wherein said water forms an aqueous phase.

77. A composition according to claim 76, wherein said aqueous phase comprises at least one additional film-forming polymer in a form of particles dispersed in said aqueous phase.

78. A composition according to claim 1, wherein said composition is in the form of a fluid, a gel, a semi-solid, a soft paste or a solid.

79. A composition according to claim 1, wherein said composition is in the form of a composition for making up or cosmetically treating at least one keratinous substance.

80. A composition according to claim 79, wherein said composition for making up or cosmetically treating at least one keratinous substance is chosen from compositions for caring for skin.

81. An eyeliner product, a product for coating keratinous fibres, a foundation product, an eyeshadow product, a blusher product, a lipstick product, a concealer product or a product for making up the body of the temporary or semi-permanent tattooing type comprising at least one liquid fatty phase comprising:
(a) at least one oil; and
(b) at least one wax having a melting point of less than 60° C. and a hardness at 20° C. of greater than or equal to 9.5 MPa.

82. A process for caring for, making up or treating at least one keratinous substance comprising applying to said at least one keratinous substance a composition comprising at least one liquid fatty phase comprising:
(a) at least one oil; and
(b) at least one wax having a melting point of less than 60° C. and a hardness at 20° C. of greater than or equal to 9.5 MPa.

83. A process for non-therapeutic treatment of at least one keratinous substance comprising applying to said at least one keratinous substance a composition comprising at least one liquid fatty phase comprising:
(a) at least one oil; and
(b) at least one wax having a melting point of less than 60° C. and a hardness at 20° C. of greater than or equal to 9.5 MPa.

84. A process for curving keratinous fibres comprising applying to said keratinous fibres a composition comprising at least one liquid fatty phase comprising:
(a) at least one oil; and
(b) at least one wax having a melting point of less than 60° C. and a hardness at 20° C. of greater than or equal to 9.5 MPa.

85. A process according to claim 84, wherein said keratinous fibres are eyelashes.
